SEQUENCE LISTING (48 sequences)

SEQ ID NO: 1 - DNA, 20 bp, Artificial Sequence
  Synthetic primer
cactggctcactgaactgga

SEQ ID NO: 2 - DNA, 20 bp, Artificial Sequence
  Synthetic primer
aaagtggggaaaatggaagc

SEQ ID NO: 3 - DNA, 20 bp, Artificial Sequence
  Synthetic primer
caaggccctgtctctactgc

SEQ ID NO: 4 - DNA, 20 bp, Artificial Sequence
  Synthetic primer
cgtcaatggacatctggttg

SEQ ID NO: 5 - DNA, 20 bp, Artificial Sequence
  Synthetic primer
ccttctgaggtccgagactg

SEQ ID NO: 6 - DNA, 20 bp, Artificial Sequence
  Synthetic primer
aaagatctccagctccgtca

SEQ ID NO: 7 - DNA, 20 bp, Artificial Sequence
  Synthetic primer
cccacttctgctacctgctc

SEQ ID NO: 8 - DNA, 20 bp, Artificial Sequence
  Synthetic primer
ccgcgatctttctcgtagtc

SEQ ID NO: 9 - DNA, 20 bp, Artificial Sequence
  Synthetic primer
agaatgggaccacaaacgag

SEQ ID NO: 10 - DNA, 20 bp, Artificial Sequence
  Synthetic primer
tgagggtcatcatcactgga

SEQ ID NO: 11 - DNA, 20 bp, Artificial Sequence
  Synthetic primer
gaccgtcgttgccttgtatt

SEQ ID NO: 12 - DNA, 20 bp, Artificial Sequence
  Synthetic primer
atgccaaattcttggtgagg

SEQ ID NO: 13 - DNA, 20 bp, Artificial Sequence
  Synthetic primer
tgacgtggcctaccataaca

SEQ ID NO: 14 - DNA, 20 bp, Artificial Sequence
  Synthetic primer
ggatgcggtcggagtagtta

SEQ ID NO: 15 - DNA, 20 bp, Artificial Sequence
  Synthetic primer
atcaccttgctgtgggtttc

SEQ ID NO: 16 - DNA, 20 bp, Artificial Sequence
  Synthetic primer
ctgagcatcaggctgtacca

SEQ ID NO: 17 - DNA, 20 bp, Artificial Sequence
  Synthetic primer
gtcactaccacgccaatgtg

SEQ ID NO: 18 - DNA, 20 bp, Artificial Sequence
  Synthetic primer
ttcggtcggaatagttgtcc

SEQ ID NO: 19 - DNA, 20 bp, Artificial Sequence
  Synthetic primer
catcctggtgtgtccaatca

SEQ ID NO: 20 - DNA, 20 bp, Artificial Sequence
  Synthetic primer
atttttccacggaagcattg

SEQ ID NO: 21 - DNA, 20 bp, Artificial Sequence
  Synthetic primer
ttcaaccgaaatgacgaaca

SEQ ID NO: 22 - DNA, 20 bp, Artificial Sequence
  Synthetic primer
cagcaatcagcaatgcaagt

SEQ ID NO: 23 - DNA, 20 bp, Artificial Sequence
  Synthetic primer
ctcctgaccattgggcttta

SEQ ID NO: 24 - DNA, 20 bp, Artificial Sequence
  Synthetic primer
ccagctcctcttcctcacac

SEQ ID NO: 25 - DNA, 20 bp, Artificial Sequence
  Synthetic primer
agcaaagggtaccggagaat

SEQ ID NO: 26 - DNA, 20 bp, Artificial Sequence
  Synthetic primer
tccagggacaggtactccac

SEQ ID NO: 27 - DNA, 20 bp, Artificial Sequence
  Synthetic primer
cattgctcaggaaatgctca

SEQ ID NO: 28 - DNA, 20 bp, Artificial Sequence
  Synthetic primer
tggtgcctctgtggtcaata

SEQ ID NO: 29 - DNA, 20 bp, Artificial Sequence
  Synthetic primer
atcctgaggggcttcaaact

SEQ ID NO: 30 - DNA, 23 bp, Artificial Sequence
  Synthetic primer
tcactctggatgtgcttacaaga

SEQ ID NO: 31 - DNA, 20 bp, Artificial Sequence
  Synthetic primer
gccattagaaagcaggcaac

SEQ ID NO: 32 - DNA, 20 bp, Artificial Sequence
  Synthetic primer
ctctctgcagtcccttccag

SEQ ID NO: 33 - DNA, 20 bp, Artificial Sequence
  Synthetic primer
tataccacaagcccctctgc

SEQ ID NO: 34 - DNA, 20 bp, Artificial Sequence
  Synthetic primer
atcgccagtgatgattctcc

SEQ ID NO: 35 - DNA, 20 bp, Artificial Sequence
  Synthetic primer
gcggcaagaaggtagcagta

SEQ ID NO: 36 - DNA, 20 bp, Artificial Sequence
  Synthetic primer
agttgtgatcgcacctctcc

SEQ ID NO: 37 - DNA, 20 bp, Artificial Sequence
  Synthetic primer
cctgcgacagagctggttat

SEQ ID NO: 38 - DNA, 20 bp, Artificial Sequence
  Synthetic primer
cacgtggtcatcgcttgtta

SEQ ID NO: 39 - DNA, 20 bp, Artificial Sequence
  Synthetic primer
gctctctccagagaccatcg

SEQ ID NO: 40 - DNA, 20 bp, Artificial Sequence
  Synthetic primer
atctgcttgaacccatcagg

SEQ ID NO: 41 - DNA, 20 bp, Artificial Sequence
  Synthetic primer
tcaggtgcaggtatgcagag

SEQ ID NO: 42 - DNA, 20 bp, Artificial Sequence
  Synthetic primer
gttgaccatctgcaccacac

SEQ ID NO: 43 - DNA, 20 bp, Artificial Sequence
  Synthetic primer
ttttgtggacttggcatcaa

SEQ ID NO: 44 - DNA, 20 bp, Artificial Sequence
  Synthetic primer
cttggaaggcattgttggtt

SEQ ID NO: 45 - DNA, 20 bp, Artificial Sequence
  Synthetic primer
tgtttcgaatcaagcagcac

SEQ ID NO: 46 - DNA, 20 bp, Artificial Sequence
  Synthetic primer
catctttcctcctccccatt

SEQ ID NO: 47 - DNA, 20 bp, Artificial Sequence
  Synthetic primer
atgcacctgtacgatcactg

SEQ ID NO: 48 - DNA, 21 bp, Artificial Sequence
  Synthetic primer
acaaaggacatggagaacacc

(19) United States
(12) Patent Application Publication
(10) Pub. No.: US 2013/0225597 A1
(43) Pub. Date: Aug. 29, 2013

(54) USE OF PHOSPHODIESTERASE INHIBITOR TO ENHANCE POST-SURGICAL ERECTION IN MEN UNDERGOING RADICAL PROSTATECTOMY

(71) Applicant: University of Medicine and Dentistry of New Jersey, (US)

(72) Inventors: Isaac Yi Kim, Belle Meade, NJ (US); Matthew C. Ercolani, Englewood, FL (US)

(73) Assignee: University of Medicine and Dentistry of New Jersey, Somerset, NJ (US)

(21) Appl. No.: 13/872,525

(22) Filed: Apr. 29, 2013

Related U.S. Application Data
(63) Continuation-in-part of application No. PCT/US2011/058354, filed on Oct. 28, 2011.
(60) Provisional application No. 61/408,201, filed on Oct. 29, 2010.

(51) Int. Cl. A61K 9/00; A61K 31/519

(52) U.S. Cl. CPC A61K 9/0014; A61K 31/519
USPC 514/252.16

(57) ABSTRACT

The present invention is a method for treating post-radical prostatectomy erection dysfunction in a patient using a PDE-4, PDE-5, PDE-6 and/or PDE-7 inhibitor, wherein said PDE inhibitor is delivered at the incision site via an adhesion barrier.

This application is a continuation-in-part application of PCT/US2011/058354, filed Oct. 28, 2011, which claims the benefit of priority from U.S. Patent Application Ser. No. 61/408,201, filed Oct. 29, 2010, the contents of which are incorporated herein by reference in their entireties.

### BACKGROUND OF THE INVENTION

Each year, approximately 100,000 men undergo radical prostatectomy in the United States. Erectile dysfunction, or impotence, is a frequently encountered complication of the surgery. Following prostate cancer surgery, most patients experience impotence (10-50%) either temporarily or permanently because the cavernosal nerves that control erection are invariably damaged; these nerves run very close to the prostate such that prostate cancer surgery cannot be done without manipulating the nerves.

The cavernosal nerves are damaged either by traction or transaction injury. Basic science research supports the concept that erectile loss after pelvic surgery is frequently related to neuropathic effects. This injury in turn results in local inflammation and fibrosis, which is another major mechanism of erectile dysfunction following surgery. Regardless of the mechanism of nerve injury, the cavernosal nerves are fully exposed during prostate cancer surgery. Thus, the optimal time to prevent further nerve damage and to rehabilitate the cavernosal nerves is during surgery.

Potency is generally defined as the ability to have unassisted intercourse. In 2000, a John Hopkins' study showed potency was achieved in 38%, 54%, and 73% of sixty-four patients three months, six months and twelve months postoperative radical prostatectomy, respectively (Walsh, et al. (2000) Urology 55(1):58-61). A previous John Hopkins study from 1991, showed 76% recovery of potency of 291 men by eighteen months (Quinlan, et al. (1991) J. Urol. 145(5):998-1002). A study of 164 men following radical prostatectomy showed a potency recovery of 40% at twelve months and 58% at twenty-three months (Touijer, et. al. (2008) J. Urol. 179(5):1811-7).

There are a few good options for men suffering from erectile dysfunction following prostatic surgery. A well-publicized oral medication, sildenafil citrate, sold under the trademarked name VIAGRA, is available. Sildenafil citrate, a phosphodiesterase-5 (PDE-5) inhibitor, has been approved by the United States Food and Drug Administration (FDA) to treat erectile dysfunction in men. The use of daily oral sildenafil citrate has been recommended by many experts post radical prostectomy. Despite improvement in the rate of recovery with daily dosing, complete recovery usually takes at least one year. Thus, additional agents that may improve the rate as well as speed of recovery of sexual function following prostate cancer surgery are needed.

Formulations of sildenafil citrate, either exclusively or in combination with other medications, have been disclosed in U.S. Pat. No. 6,200,591. This document indicates that administration is limited to oral, enteral, or intranasal routes, thereby requiring the drug to travel systemically prior to reaching the targeted area, smooth muscle cells.

It has been suggested that sildenafil citrate may treat erectile dysfunction by acting on the central nervous system versus its current use to relax smooth muscle. The results suggest that PDE-5 inhibitors may increase sexual arousal by activating the mesolimbic dopaminergic neurons Sanna, et al. (2009) J. Sex. Med. 6(10):2680-89). However, these studies have failed to focus on treating post-surgical erectile dysfunction caused by neuron apoptosis and local inflammation and fibrosis.

Studies have shown that selective PDE-5 inhibitors, specifically dipyridamole, T-1032, and zaprinas, protect spinal motor and non-motor neurons against acute Reactive Oxygen Species-induced neurotoxicity (Nakamizo, et al. (2003) J. Neurosci. Res. 71(4):485-95). The study did not address PDE-5 inhibitors application on cavernosal neurons during prostate cancer surgery. In addition, this study did not suggest sildenafil citrate or the other marketed PDE-5 inhibitors, vardenafil (trademarked LAVITRA) and tadalifil (trademarked CIALIS), as substitutes for the studied PDE-5 inhibitors.

It has been established that PDE-5 inhibitors have different potency and selectivity for target sites. Amongst the FDA-approved PDE-5 inhibitors only tadalifil cross-reacts with PDE-11; whereas, vardenafil and sildenafil citrate are selective for PDE-6. PDE-6 is important to phototransduction cascade. Sildenafil citrate has greater selectivity than vardenafil for PDE-6δ (rods), whereas the converse was true for PDE-6 (cones)(Gresser (2002) Eur. J. Med. Res. 7:435-46). Thus, side effects from inhibition of PDE-6, likely to be shared by vardenafil and sildenafil citrate, may include visual impairment (McCullogh (2003) J. Andrology 24(6 Supp):S52-S58). Accordingly, the pharmacodymanic variations of PDE-5 inhibitors may yield different clinical results.

In addition to the neuronal injury, adhesions are an auxiliary mechanism of erectile dysfunction following prostatectomy. Adhesions are the leading cause of post-surgical complications. Adhesions are fibrous bands that form between tissues because of surgical injury. They formed as a result of the body's natural healing process following trauma, such that the tissues will adhere together and form fibrous scar tissue.

Approaches to preventing adhesions include use of anti-inflammatory agents, anticoagulants agents and fibrinolytic agents to reduce the inflammatory response during surgery. However, these agents have had minimal effect on reducing adhesions. Additionally, reabsorbable liquid barriers have been marketed to reduce adhesions. Research has shown that that reabsorption time of approximately four weeks is less than ideal in the prevention of adhesions. However, structural barriers, particularly bioasorbable, when placed between layers of traumatized tissues have shown marked improvement in preventing adhesions.

GYNECARE INTERCEED absorbable adhesion barrier has an indicated use in reducing postoperative pelvic adhesions following open gynecologic pelvic microsurgical procedures. GYNECARE INTERCEED absorbable adhesion barrier has not been approved for other surgical procedures and may increase the risk of adhesions if misapplied. Such misapplications include folding, wading or layering of the barrier. In addition, as this barrier is not a hemostatic agent, appropriate means must be employed to achieve hemostasis or postoperative adhesions may be induced.

SEPRAFILM adhesion barrier is a biodegradable sheet that has been approved by the FDA for the prevention of fibrosis and adhesions in open abdominal or gynecologic surgeries including a C-section, hysterectomy, myomectomy, colectomy or hernia repair. A recent study evaluated the safety of SEPRAFILM adhesion barrier in proximity to peripheral nerve tissue (Magill, et. al. (2009) J. Reconstr. Microsurg. 25(6):345-54). It was noted that application of SEPRAFILM adhesion barrier resulted in qualitatively fewer scar bands and scar tissue on cut and repaired neurons, though no differences in functional outcomes were detected. In addition, no deleterious effects were noted from placing SEPRAFILM adhesion barrier on nerves. However, this study did not address the use of SEPRAFILM or any adhesion barrier directly on nerve bundles to minimize inflammation and fibrosis following prostate cancer surgery.

### SUMMARY OF THE INVENTION

The present invention fulfills the foregoing need by providing a method for treating post-radical prostatectomy erection dysfunction in a subject by administering a therapeutically effective amount of a phosphodiesterase (PDE) inhibitor or a pharmaceutically acceptable salt or solvate thereof at the site of incision for prostatectomy. The present invention is based upon the discovery that inhibition of PDE enhances post-surgical erection in men following radical prostatectomy. The utility of inhibition of PDE enhances post-surgical erection in men following radical prostatectomy using pharmaceutical agents is demonstrated. In some embodiments of the instant method, the PDE inhibitor is a PDE-5 inhibitor with selectivity for PDE-4δ, PDE-6δ and/or PDE-7α. In other embodiments, the PDE inhibitor is a PDE-4δ, PDE-6δ or PDE-7α inhibitor. In particular embodiments, the PDE inhibitor is sildenafil citrate. Carriers for delivering the PDE inhibitor are also provided. Such carriers include aqueous carriers, matrices, or absorbable adhesive barriers such as a cellulose absorbable adhesion barrier, which can be cut into pieces and delivered with normal saline.

### BRIEF DESCRIPTION OF DRAWINGS

FIG. 1 shows a comparison of neuronal viability of hydrogen peroxide treated PC-12 cells following administration of sildenafil citrate, vardenafil, and tadalafil at 0.1-fold physiologic dose (P.D.) (0.5 μM, 0.3 μM and 0.4 μM, respectively), physiologic dose (5 μM, 3 μM and 4 μM, respectively) and 10-fold physiologic dose (50 μM, 30 μM and 40 μM, respectively) as compared to control cells not treated with a PDE inhibitor.

FIG. 2 shows a comparison of neuronal viability of hydrogen peroxide treated PC-12 cells following administration of sildenafil citrate (FIG. 2A), tadalafil (FIG. 2B), and vardenafil (FIG. 2C) at the levels indicated as compared to control (C) and H₂O₂ (H).

FIG. 3 shows the effect of PDE-4 inhibitor, rolipram, on hydrogen peroxide toxicity in PC-12 cells as compared to control (C) and H₂O₂ (H).

### DETAILED DESCRIPTION OF THE INVENTION

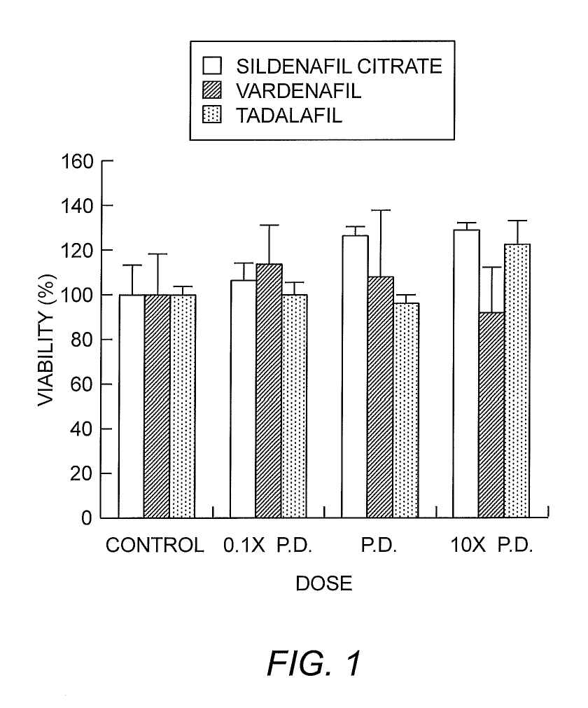

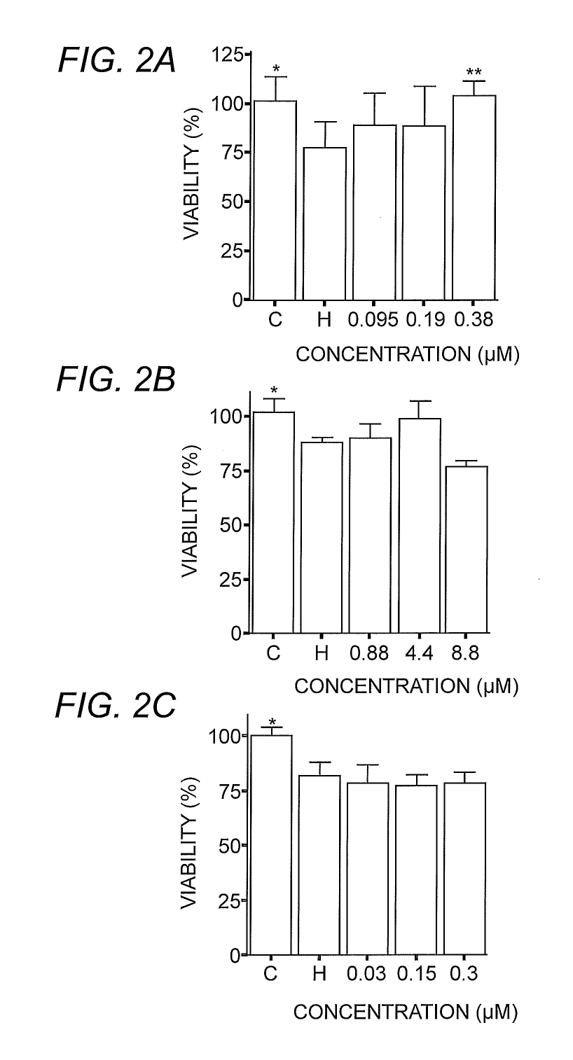

The invention provides a method of improving potency recovery following radical prostatectomy by administering a therapeutically effective amount of one or more PDE-4, PDE-5, PDE-6 and/or PDE-7 inhibitor(s). It has now been shown that the discovery that sildenafil citrate, as compared to other PDE-5 inhibitors, has a neuronal cell protective effect. In particular, the present experiments compared viability of PC-12 neuronal cells in the presence of purified sildenafil citrate, vardenafil, and tadalafil following H₂O₂-induced apoptosis. As shown in FIGS. 1 and 2, sildenafil citrate protected against cell death in a significant manner.

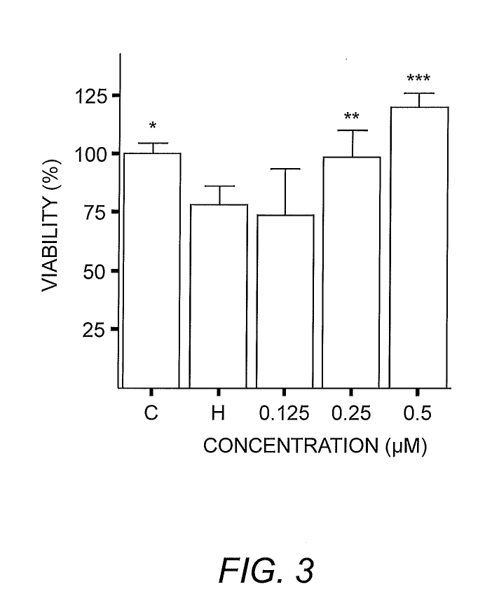

Further experimentation on mechanism of action showed that PDE-5, the traditional target of sildenafil citrate, vardenafil and tadalafil, is not present in neuronal cells. Therefore, the aforementioned protective effect of sildenafil citrate on neuronal cells is not via targeting of PDE-5. Experiments with neuronal cells indicated that PDE-4δ, PDE-6δ and PDE-7α are the targets for neuronal protection. Thus, sildenafil citrate's lack of discrimination for PDE-5 versus PDE-4δ, PDE-6δ and PDE-7α, as compared to the other PDE-5 inhibitors, may mediate the potent neuronal protection. Thus, true PDE-4δ, PDE-6δ and PDE-7α inhibitors may achieve optimal protection. In this respect, PDE-4 inhibitor, rolipram was shown to provide significant protection against H₂O₂-induced cell death (FIG. 3).

Accordingly, the present invention features a method for treating post-radical prostatectomy erection dysfunction in a subject by administering to a subject in need of treatment a therapeutically effective amount of a PDE inhibitor or a pharmaceutically acceptable salt or solvate thereof at the site of incision for prostatectomy. For the purposes of the present invention, the term “therapeutically effective amount” represents an amount of a compound that is capable of inhibiting PDE-4δ, PDE-5, PDE-6δ or PDE-7α and causes a measurable improvement in clinical recovery of potency following radical prostatectomy. Such improvement includes at least a 15, 20, 25, 30, or 35% improvement in potency at three months post-operative compared to a subject not receiving treatment; at least a 30, 35, 40, 45, 50, 55, 60, 65, 70, 75, 80 or 85% improvement in potency at six months post-operative compared to a subject not receiving treatment; or at least a 75, 80, 85, 90, or 95% improvement in potency at twelve months post-operative compared to a subject not receiving treatment.

The term “PDE inhibitor” refers to a compound that inhibits PDE. A PDE inhibitor useful in the present invention is a compound that inhibits PDE-4, PDE-5, PDE-6 and/or PDE-7. Particular PDE inhibitors useful in the present invention are compounds that inhibit PDE-4, PDE-6 and/or PDE-7. More preferred PDE inhibitors are non-selective PDE-5 inhibitors with respect to PDE-4, PDE-6, and/or PDE-7, particularly PDE-4δ (also known as PDE-4D), PDE-6δ (also known as PDE-6D) and/or PDE-7α (also known as PDE-7A). Alternatively stated, particular embodiments of the present invention include the use of a PDE inhibitor that inhibits PDE-5 as well as PDE-4δ, PDE-6δ and/or PDE-7α. Particularly preferred PDE inhibitors useful in the present invention are compounds that inhibit PDE-4δ, PDE-6δ and/or PDE-7α.

PDE-5 inhibitors of use in the present invention include sildenafil citrate (trademarked VIAGRA), varedafil (trademarked LEVITRA), tadalafil (trademarked CIALIS), lodenafil (e.g., the dimer, lodenafil carbonate), mirodenafil (trademarked MVIX TAB or MVIX SODF), udenafil (trademarked ZYDENA), avanafil (trademarked STENDRA), acetildenafil, aildenafil, dipyridamole, icarin, MY-5445, nitrosoprodenafil, sulfoaildenafil, benzamidenafil, T-0156 and zaprinast. PDE-5 inhibitors, which also inhibit PDE-6, include sildenafil, vardenafil, benzamidenafil, acetildenafil, Thiosildenafil and zaprinast. See, e.g., Zou et al. (2008) J Pharm. Biomed. Anal. 47: 255-9; Venhuis, et al. (2007) Recent Developments in Counterfeits and Imitations of Viagra, Cialis and Levitra. RIVM Report 370030001/2007. National Institute for Public Health and the Environment, The Netherlands; and WO 2002/102802. Preferred PDE-5 inhibitors include sildenafil citrate; varedafil; physiologically acceptable salts and solvates thereof; and mixtures thereof. The compounds are particularly advantageous due to their ability to inhibit PDE-5 and PDE-6. Most preferred compound include sildenafil citrate; physiologically acceptable salts and solvates thereof; and mixtures thereof due to the compound's increased selectivity for PDE-6δ as compared to the other two major PDE-5 inhibitors on the market.

PDE-4 inhibitors of use in this invention include, but are not limited to, apremilast, mesembrine, S- or R-rolipram, ibudilast, piclamilast, luteolin, roflumilast (trademarked DALIRESP), cilomilast, CDP840 (R-[+]-4-[2-{3-cyclopentyloxy-4-methoxyphenyl}-2-phenylethyl]pyridine), CT-2450 (R)—N-[4-[1-(3-cyclopentyloxy-4-methoxyphenyl)-2-(4-pyridyl)ethyl]phenyl]N′-ethylurea), PMNPQ (6-(4-pyridylmethyl)-8-(3-nitrophenyl)quinoline), Ro 20-1724 (4-(3-butoxy-4-methoxyphenyl)methyl-2-imidazolidone), AWD 12-281 (N-(3,5-dichloropyrid-4-yl)-[1-(4-fluorobenzyl)-5-hydroxy-indole-3-yl]-glyoxylic acid amide), 3-cyclopentyloxy-4-methoxybenzaldehyde (GEBR-7b), NVP-ABE171 (4-(8-benzo[1,2,5]oxadiazol-5-yl-[1,7]naphthyridin-6-yl)-benzoic acid) and diazepam. In particular embodiments, the inhibitor is selective for PDE-4δ. Examples of inhibitors selective for PDE-4δ include GEBR-7b, cilomilast and NVP-ABE171 (Bruno, et al. (2011) Br. J. Pharmacol. 164:2054-63; Brown (2007) Int. J. Chron. Obstruct. Pulmon. Dis. 2:517-33; Trifilieff, et al. (2002) J. Pharmacol. Exp. Ther. 310:241-8).

An exemplary PDE-6 inhibitor of use in this invention includes, but are not limited to, zaprinast. Further exemplary compounds for use in the present invention include the thiophene-, furan- and pyrrole-containing PDE-6δ inhibitors described in US 2004/0242673 and U.S. Pat. No. 7,323,490.

PDE-7 inhibitors of use in this invention include, but are not limited to, BRL-50481 (N,N,2-Trimethyl-5-nitro-benzenesulfonamide), S14 (phenyl-2-thioxo-(1H)-quinazolin-4-on), ASB16165 (1-cyclohexyl-N-(6-(4-hydroxy-1-piperidinyl)-3-pyridinyl)-3-methyl-1H-thieno(2,3-c)pyrazole-5-carboxamide), PF0332040 (4-(5-(3-hydroxycyclohexylimino)-4-methyl-4,5-dihydro-(1,3,4)thiadiazol-2-yl)benzamide) and VP1.15 (5-(2-Hydroxyethylimino)-2,3-diphenyl-2,5-dihydro-1,2,4-thiadiazole hydrobromide) as well as pyridinylpyrazolopyrimidinone derivatives described in U.S. Pat. No. 7,943,624; imidazopyridazinones described by Banerjee, et al. (2012) Bioorg. Med. Chem. Lett. 22:6286-91; 5-imino-1,2,4-thiadiazoles described in WO 2011/0394903. In particular embodiments, the inhibitor is selective for PDE-7α. An example of an inhibitor selective for PDE-7α is ASB16165. Another exemplary compound of use in this invention is T-2585 ((2-{4-2,3-bis(hydroxymethyl)-6,7-diethoxy-1-naphthalenyl}-2-pyridinyl]-4-(3-pyridinyl)-1(2H)-phthalazinone), which inhibits both PDE-4 and PDE-7 (Nakata, et al. (2002) Clin. Exp. Immunol. 228:460-6).

During radical prostatectomy, the cavernosal nerves that control erection are invariably damaged. Thus, the optimal time to prevent further damage and to rehabilitate nerves is during surgery (e.g., during open, laparoscopic or robotic surgery). As such, use of PDE-4, PDE-5, PDE-6 and/or PDE-7 inhibitor(s) at the site of incision will preserve neuronal viability and ultimately enhance post-surgical erection in men undergoing radical prostatectomy. Therefore, in particular embodiments, the instant method is carried out during radical prostatectomy at the site of incision. Delivery of PDE-4, PDE-5, PDE-6 and/or PDE-7 inhibitor(s) includes the use of an aqueous pharmaceutical carrier, e.g., saline. A preferred mode of delivery includes time-dependent delivery of PDE-4, PDE-5, PDE-6 and/or PDE-7 inhibitor(s) at the incision site. In this respect, certain embodiments include the use of a general carrier or matrix to allow for time-dependent delivery. Such matrices are well-known in the art and routinely used in the preparation of pharmaceutical compositions.

It is contemplated that the PDE-4, PDE-5, PDE-6 and/or PDE-7 inhibitor(s) may be concurrently administrated with other carriers or agents, including other known anti-adhesion drugs or agents, anti-inflammatory agents, anti-coagulants agents, or fibrinolytic agents. Most preferably in this embodiment the pharmaceutical carrier of the PDE-4, PDE-5, PDE-6 and/or PDE-7 inhibitor(s) is an absorbable adhesive barrier, such as an absorbable cellulose adhesion barrier. Such barriers are available in the art and examples include but are not limited to SEPRAFILM adhesive barrier (hyaluronic acid-carboxymethylcellulose adhesion barrier) or GYNECARE INTERCEED absorbable adhesion barrier (heparin-saturated oxidized regenerated cellulose absorbable adhesion barrier). Delivery via an absorbable adhesive barrier provides for separation of the neurovascular bundles (NVB) from adjacent inflammatory tissue at the time of prostatectomy thereby resulting in reduced fibrosis, inflammation, and scarring and promoting earlier return of erectile function after surgery. Indeed, sexual potent patients undergoing this treatment in conjunction with intrafascial bilateral NVB sparing demonstrated a statistically significantly earlier return of potency (35% greater than controls) after RARP.

Those of ordinary skill in the art will readily optimize effective dosages and administration regimes as determined by good medical practice and clinical condition of the individual patient. In addition, most of the compounds employed in the invention can be formulated to provide quick, sustained or delayed release of the active ingredient following administration by procedures known in the art.

Having demonstrated that PDE-4δ, PDE-6δ and PDE-7α inhibitors provide a neuronal cell protective effect, it is contemplated that PDE-4δ, PDE-6δ and/or PDE-7α can used in screening assays for identifying other compounds useful in treating post-radical prostatectomy impotence.

The invention is described in greater detail by the following non-limiting examples.

### EXAMPLE 1

### Neuronal Protective Studies

Neuronal Survival Experiments. For survival experiments, PC12 neuronal cell were seeded in 6-well plates at a density of 1×10⁵ per well in Dulbecco's modified Eagle's medium (DMEM) supplemented with 1% fetal bovine serum (FBS) and 100 ng/ml nerve growth factor (NGF), herein after (“Media”), for 3 days. Media was changed every 3 days. Cells were treated in the presence or absence of 1 mM hydrogen peroxide (H₂O₂) for 1 hour in the presence or absence of either sildenafil Citrate, vardenafil, or tardalafil at 0.1-fold physiologic dose (0.5 μM, 0.3 μM and 0.4 μM, respectively), physiologic dose (5 μM, 3 μM and 4 μM, respectively) and 10-fold physiologic dose (50 μM, 30 μM and 40 μM, respectively). Treated cells were incubated for 24 hours at 37° C. Viable cells were detached and counted using an automated counter. The results of this analysis are presented in FIG. 1. Additional dose response analyses are presented in FIG. 2.

Molecular Target Analysis. For molecular target analysis, ribonucleic acid (RNA) was isolated using a commercially available agent (TRIZOL; Invitrogen, Carlsbad, Calif.) per the manufacturer's protocol.

For polymerase chain reaction (PCR) amplifications, RNA was brought to final 25 μl reaction volume containing 0.2 μl of Taq polymerase, 10 pmol of each primer, 0.75 mM MgCl₂, 200 μM each dNTP, and 2.5 μl of 10× reaction buffer (supplied by manufacturer). The amplification cycle included denaturing at 95° C. for 30 seconds, annealing at 55° C. for 30 seconds, and extending at 72° C. for 30 seconds in a Thermal Cycler. PCR was carried out for 35 cycles. After PCR, the DNA products were analyzed by electrophoresis in 0.7% agarose gels.

Oligonucleotides of use in the amplification of members of the PDE super family in humans are listed in Table 1.

TABLE 1

| | | Primer Sequence | SEQ ID |
|---|---|---|---|
| PDE Member | Primer | 5′−> 3′ | NO: |
| hPDE1A | Forward | CACTGGCTCACTGAACTGGA | 1 |
| | Reverse | AAAGTGGGGAAAATGGAAGC | 2 |
| hPDE1B | Forward | CAAGGCCCTGTCTCTACTGC | 3 |
| | Reverse | CGTCAATGGACATCTGGTTG | 4 |
| hPDE1C | Forward | CCTTCTGAGGTCCGAGACTG | 5 |
| | Reverse | AAAGATCTCCAGCTCCGTCA | 6 |
| hPDE2A | Forward | CCCACTTCTGCTACCTGCTC | 7 |
| | Reverse | CCGCGATCTTTCTCGTAGTC | 8 |
| hPDE3A | Forward | AGAATGGGACCACAAACGAG | 9 |
| | Reverse | TGAGGGTCATCATCACTGGA | 10 |
| hPDE3B | Forward | GACCGTCGTTGCCTTGTATT | 11 |
| | Reverse | ATGCCAAATTCTTGGTGAGG | 12 |
| hPDE4A | Forward | TGACGTGGCCTACCATAACA | 13 |
| | Reverse | GGATGCGGTCGGAGTAGTTA | 14 |
| hPDE4B | Forward | ATCACCTTGCTGTGGGTTTC | 15 |
| | Reverse | CTGAGCATCAGGCTGTACCA | 16 |
| hPDE4C | Forward | GTCACTACCACGCCAATGTG | 17 |
| | Reverse | TTCGGTCGGAATAGTTGTCC | 18 |
| hPDE4D | Forward | CATCCTGGTGTGTCCAATCA | 19 |
| | Reverse | ATTTTTCCACGGAAGCATTG | 20 |
| hPDE5A | Forward | TTCAACCGAAATGACGAACA | 21 |
| | Reverse | CAGCAATCAGCAATGCAAGT | 22 |
| hPDE6A | Forward | CTCCTGACCATTGGGCTTTA | 23 |
| | Reverse | CCAGCTCCTCTTCCTCACAC | 24 |
| hPDE6B | Forward | AGCAAAGGGTACCGGAGAAT | 25 |
| | Reverse | TCCAGGGACAGGTACTCCAC | 26 |
| hPDE6C | Forward | CATTGCTCAGGAAATGCTCA | 27 |
| | Reverse | TGGTGCCTCTGTGGTCAATA | 28 |
| hPDE6D | Forward | ATCCTGAGGGGCTTCAAACT | 29 |
| | Reverse | TCACTCTGGATGTGCTTACAAGA | 30 |
| hPDE7A | Forward | GCCATTAGAAAGCAGGCAAC | 31 |
| | Reverse | CTCTCTGCAGTCCCTTCCAG | 32 |
| hPDE7B | Forward | TATACCACAAGCCCCTCTGC | 33 |
| | Reverse | ATCGCCAGTGATGATTCTCC | 34 |
| hPDE8A | Forward | GCGGCAAGAAGGTAGCAGTA | 35 |
| | Reverse | AGTTGTGATCGCACCTCTCC | 36 |
| hPDE8B | Forward | CCTGCGACAGAGCTGGTTAT | 37 |
| | Reverse | CACGTGGTCATCGCTTGTTA | 38 |
| hPDE9A | Forward | GCTCTCTCCAGAGACCATCG | 39 |
| | Reverse | ATCTGCTTGAACCCATCAGG | 40 |
| hPDE10A | Forward | TCAGGTGCAGGTATGCAGAG | 41 |
| | Reverse | GTTGACCATCTGCACCACAC | 42 |
| hPDE11A | Forward | TTTTGTGGACTTGGCATCAA | 43 |
| | Reverse | CTTGGAAGGCATTGTTGGTT | 44 |
| hPDE12 | Forward | TGTTTCGAATCAAGCAGCAC | 45 |
| | Reverse | CATCTTTCCTCCTCCCCATT | 46 |
| β-actin | Forward | ATGCACCTGTACGATCACTG | 47 |
| | Reverse | ACAAAGGACATGGAGAACACC | 48 |

Clinical Studies. For effective study in humans, 147 men that underwent robot-assisted radical prostatectomy were treated with sildenafil citrate at the operative site. Potency was reported in 24.2%, 84.2% and 93.3% of the subjects at three, six, and twelve months post-operative, respectively.

### EXAMPLE 2

### Hyaluronic Acid-Carboxymethylcellulose Adhesion Barrier Facilitates Earlier Return of Potency after Robotic Prostatectomy

Patient Selection. Hyaluronic acid-carboxymethylcellulose adhesion barrier (SEPRAFILM, Genzyme, Cambridge, Mass.) usage is approved by the FDA for the prevention of adhesions during abdominopelvic surgery. Two hundred consecutive patients underwent standard transperitoneal robotic-assisted radical prostatectomy (RARP) with or without bilateral neurovascular bundles (NVB) spared by a single surgeon. Criteria for the bilateral NVB sparing were Gleason score PSA and clinical stage Tic or less, number of cores from a 12-core prostate biopsy and no core ≧50% malignant tissue. Of the 200 patients, 158 met these criteria and underwent bilateral NVB nerve sparing. The remaining 40 patients had unilateral nerve spared along with unilateral neurovascular bundle resection. Two patients had bilateral neurovascular bundle resections. All patients completed the self-administered American Urological Association Symptom Score (AUASS) and International Index of Erectile Function (IIEF) before and after the surgery. Subgroup stratification was done to analyze patients only undergoing RARP with bilateral nerves spared (n=158), patients with an IIEF (n=87), and patients with an IIEF who underwent RARP with bilateral nerves spared (n=72).

Surgical Technique. The intrafascial nerve sparing surgical technique is known in the art (Potdevin, et al. (2009) J. Endourol. 23:1479). After completing anastomosis, 10 mL of dissolved hyaluronic acid-carboxymethylcellulose adhesion barrier was delivered laparoscopically around the left and right NVB and the lateral edge of the vesicourethral anastomosis and bladder (total =20 mL). Surgical drains were not placed in all patients. All patients were discharged home at postoperative day 1 with urethral catheters that were removed at post-operative day 7.

Clinical Follow-Up. The following perioperative data points were retrospectively collected and analyzed: patient age, operative time, estimated blood loss, pre-operative PSA, pre-operative AUASS, pre-operative IIEF, final pathologic Gleason sum, length of hospital stay, prostate weight, and surgical margin status. All patients underwent routine postoperative follow up with urethral catheter removal 7 days after surgery and detailed physical examinations. Additionally, EF assessment with IIEF was completed by the patients at 3 and 6 months. Potency was defined as the ability to achieve penetration ≧50% of the time as per questions 2 and 3 on SHIM survey.

Statistical Analysis. All statistical analysis was performed using Stata 8.0 (StataCorp LP, College Station, Tex.). A Mann-Whitney test was utilized to determine statistically significant differences between components of each arm of the study (Group 1—control and Group 2=hyaluronic acid-carboxymethylcellulose adhesion barrier use) as well as the stratified subgroups. Chi-square analysis was then used to determine odds ratios and risk differences between groups. Utilizing multivariate logistic regression analysis, independent predictors of erectile function at three and six months after surgery were determined. Results were considered significant if the p-value was

Results. No significant differences between Group 1 (control) and Group 2 (hyaluronic acid-carboxymethylcellulose adhesion barrier use) were observed in ten categories. Analyses of recovery of erectile function in the two groups at 3 and 6 months post-surgery indicated no improvement in potency in Group 2 at 3 months. However, Group 2 demonstrated a 16% improvement in potency over the control group at 6 months (p=0.01). Multivariate logistic regression analysis performed on this group at the month period demonstrated that hyaluronic acid-carboxymethylcellulose adhesion barrier use and pre-operative IIEF score were independent predictors of potency recovery (p<0.0001). The major peri-operative complication rate was 1% in each group (1 fascial dehiscence at supraumbilical incision in group 1 and 1 rectal injury in group 2). Given the significant improvement in recovery of erectile function at 6 months in Group 2, subgroups were isolated to determine if the improvement was more remarkable in patients who had better pre-operative sexual function (SHIM≧20), underwent intrafascial bilateral NVB sparing, or both (bil NVB spared+SHIM≧20). Differences between Group 2 and the bil NVB spared group demonstrate statistically equivalent (16% vs. 14%) improvement in potency at 6 months with hyaluronic acid-carboxymethylcellulose adhesion barrier use. When patients with SHIM≧20 pre-operatively were analyzed, the potency rate in the hyaluronic acid-carboxymethylcellulose adhesion barrier group at 6 months rose to 33% (p=0.002). When combined with the bil NVB spared group, this potency improvement increased slightly to 35% at 6 months with hyaluronic acid-carboxymethylcellulose adhesion barrier use (p=0.002).

In this study, the use of a hyaluronic acid-carboxymethylcellulose adhesion barrier had a statistically significant impact on early potency outcomes, with a 16% overall improvement in potency at 6 months and a 35% improvement in potency at 6 months in patients with normal erectile function who underwent our bilateral NVB sparing RARP. Most importantly, hyaluronic acid-carboxymethylcellulose adhesion barrier use is confirmed as an independent predictor for potency return.

What is claimed is:

1. A method for treating post-radical prostatectomy erection dysfunction in a subject comprising administering to a subject in need of treatment a therapeutically effective amount of a phosphodiesterase (PDE) inhibitor or a pharmaceutically acceptable salt or solvate thereof at the site of incision for prostatectomy thereby treating post-radical prostatectomy erection dysfunction in the subject.

2. The method of claim 1, wherein said PDE inhibitor is a PDE-5 inhibitor with selectivity for PDE-4δ, PDE-6δ or PDE-7α.

3. The method of claim 1, wherein said PDE inhibitor is a PDE-4δ, PDE-6δ or PDE-7α inhibitor.

4. The method of claim 1, wherein said PDE inhibitor is sildenafil citrate.

5. The method of claim 1, wherein the PDE inhibitor is administered with a carrier.

6. The method of claim 5, wherein the carrier is aqueous.

7. The method of claim 5, wherein the carrier is a matrix.

8. The method of claim 5, wherein the carrier is an absorbable adhesive barrier.

9. The method of claim 8, wherein the absorbable adhesive barrier is a cellulose absorbable adhesion barrier.

10. The method of claim 8, wherein said absorbable adhesive barrier is cut into pieces and delivered with normal saline.
